(19) United States
(12) Patent Application Publication
(10) Pub. No.: US 2002/0077689 A1
(43) Pub. Date: Jun. 20, 2002

(54) Electrode positioning bodysuit

(76) Inventor: Thomas Christopher Kirkland, Coconut Creek, FL (US)

Correspondence Address: THOMAS C. KIRKLAND, 4682 St. Simon DR., Coconut Creek, FL, 33073, US

(21) Appl. No.: 09/929,471

(22) Filed: Aug. 15, 2001

Related U.S. Application Data
(60) Provisional application No. 60/256,184, filed on Dec. 15, 2000.

(51) Int. Cl. A61N 1/04

(52) U.S. Cl. 607/149

(57) ABSTRACT

A suit of a flexible elastomeric material that fits snug to the body is used in conjunction with an electrical muscle and/or nerve stimulation device. The suit is specifically structured to advantageously position electrode pads at predetermined positions on the body corresponding with different muscle groups and is provided with pockets, rivets, thread, or adhesive for holding the electrode pads at the predetermined positions. The electrode pads are interconnected by wires, which are routed through seams in the arms, legs, back, and chest of the suit. The wires exit at the waist and lead to an interface connector that connects to output connector of the stimulation device. A color-coded connection between the output connector of the stimulation device and the interface connector enables selectively directing of stimulation signals to one or more of the electrode pads to thereby target specific muscle groups of the body. The stimulation device permits selective adjustment of the duration and intensity of the signal and electrical pulse delivered to specific muscle groups and/or nerve areas.

### BACKGROUND OF THE INVENTION

1. Field of the Invention

The present invention relates generally to Transcutaneous Electrical Nerve Stimulation (TENS) and Electrical Muscle Stimulation (EMS) devices for providing a stimulating waveform and electrical pulses to select muscle groups and nerve areas of the body and, more particularly, to a partial or full body suit which includes means for positioning a plurality of electrode pads at select, predetermined locations on the body to thereby deliver electrical pulses to targeted muscle groups and nerve areas of the body in accordance with the needs of the user.

2. Description of the Related Art

TENS and EMS devices that apply stimulation signals to particular areas of the human body in order to suppress pain or stimulate a muscle are well known in the art. It has been well documented that applying a pulsed waveform, having an adjustable intensity, pulse duration and pulse width, at an inflicted area of tissue to stimulate nerve fibers is effective to suppress pain originating for the immediate area. TENS devices have been used for symptomatic relief and management of chronic, post surgical, and traumatic acute pain. EMS devices use more powerful waveforms to cause a muscle contraction. These induced muscle contractions are useful in physical therapy and body building for correcting atrophy, stimulating muscle growth, and increasing range of motion. Studies have shown that EMS stimulates large nerve axons, some of which cannot be stimulated voluntarily. It is further believed that EMS allows for additional muscle hypertrophy (i.e., increased development of tissue by enlargement, without multiplication of cells). Throughout the following specification, TENS and EMS devices will be collectively referred to as stimulation devices. A typical stimulation unit consists of a housing, which may be carried, in a case, a plurality of electrode pads, wires connecting the one or more electrode pads to an interface connector. The interface connector plugs into output connectors on the stimulation unit.

Generally, stimulation devices include a pulse generating circuit, which generates a pulse waveform having the desired parameters. One or more electrodes positioned against a person's skin applying electrical pulses to the area at which the electrode is positioned. Each electrical pulse delivered by the electrode stimulates the targeted muscles, causing them to contract. By varying the frequency, amplitude, pulse width, time, and waveform shape of the stimulation signal, the effect of the stimulation on the nerve or muscle group can be controlled.

While the use of stimulation devices to perform TENS and EMS has been found to be highly effective in pain therapy, muscle rehabilitation and muscle development, the use of such devices can be inconvenient. Specifically, each time an EMS or TENS device is used, the patient must position the electrode pads on the body and then individually connect the electrode pads to the stimulation unit. After each treatment, the patient must then remove each of the electrode pads and disconnect the interconnecting wires. A further problem is encountered when trying to identify the specific location on the body for placement of the electrode pads. While doctors and physical therapist are able to more easily identify the optimal locations on the body for placement of the electrode pads, individuals using the stimulation device at home often find it difficult to identify the correct position for placement of the electrode pads on the body. Also, when trying to position electrode pads on hard to reach areas, such as on the back, many people require the assistance of another person.

Accordingly, there is an urgent need for a means for quickly and accurately positioning a plurality of electrode pads at select muscle groups and/or nerve areas for providing optimal and efficient muscle and nerve electrical stimulation treatment using a TENS or EMS device.

### SUMMARY OF THE INVENTION

The present invention is directed to a suit, which may cover a portion of the body or the entire body. The suit is preferably made of a flexible, rubberized (i.e. elastomeric material) such as neoprene, spandex, lycra, etc., similar to a wetsuit or downhill skier's suit which fits snug to the body. Electrode pads are placed within at positions strategically located on the suit, which correspond to particular muscle groups and/ or nerve areas which can be selectively treated. Wiring from the electrode pads is routed through seams of the legs and arms to the waist of the suit. The wiring exits at the waist in a bundle and terminates at an interface. The interface, similar to a computers serial port connection, connects to the output on a TENS unit or EMS unit.

Wiring of the suit is preferably color coded to correspond with the specific areas of the body. For example, red wires may be connected to electrodes positioned on the arms, blue wires can be connected to electrodes on the legs, green wires can be connected to electrodes positioned on the abdomen muscles, orange wires can be connected to electrodes on the back, and black wires can be connected to electrodes positioned on the chest muscles. Color coding the wires in this manner enables the user to identify the particular connection of the interface connected with the output connectors of the TENS or EMS unit. This permits operation of the TENS or EMS unit to deliver electrical signals to one or more selected color coded wiring groups, thereby targeting specific muscle groups for a workout. An EMS unit used in conjunction with the suit of the present invention may be adapted to allow for selective control of electrical signal pulse; each muscle group can be specifically treated in accordance with the needs of the user.

### BRIEF DESCRIPTION OF THE DRAWINGS

For a full understanding of the nature of the present invention, reference should be made to the following description take in conjunction with the accompanying drawings, in which:

FIG. 1 is a front plan view of the electrode positioning body suit of the present invention;

FIG. 2 is a back plan view of the electrode positioning body suit;

FIG. 3 is a front perspective view of the electrode positioning body suit shown being worn on a user;

FIG. 4 is a sectional view taken along the plane of FIG. 3 showing a plurality of positions on the suit for holding electrodes against the body in accordance with select muscle group and nerve areas; and

FIG. 5 is a plan of the invention used as an abdominal wrap.

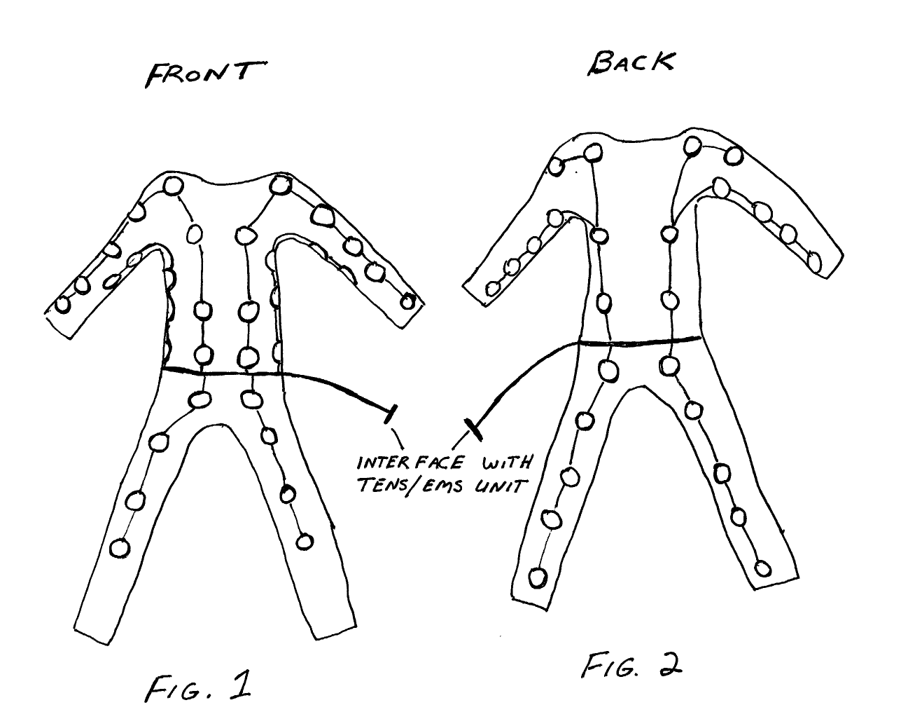

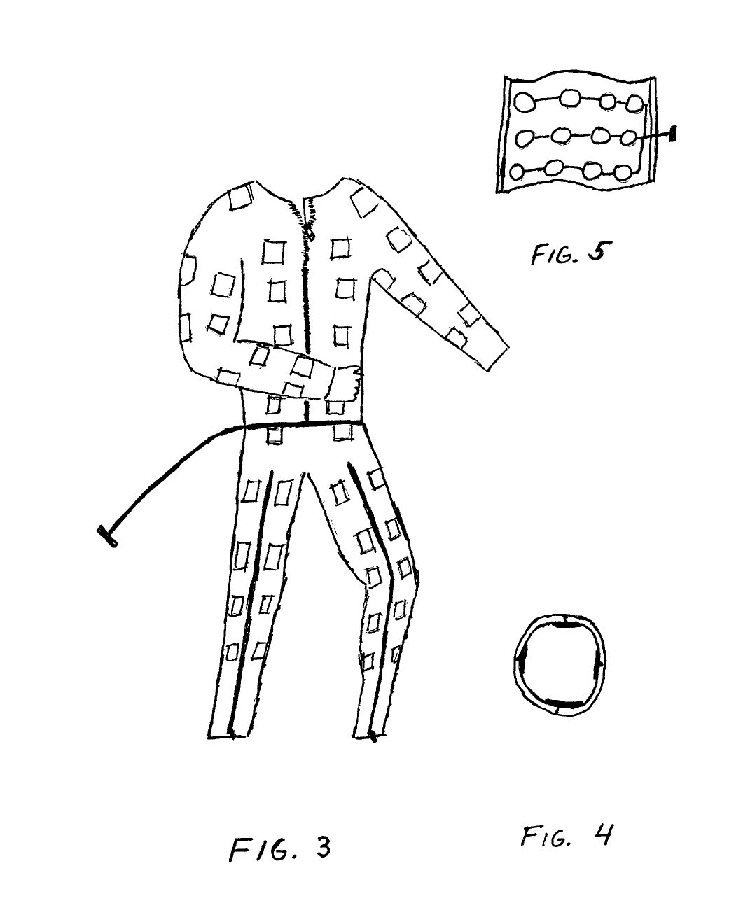

Like reference numerals refer to like part throughout the several views of the drawings.

### DETAILED DESCRIPTION OF THE PREFERRED EMBODIMENT

Referring to the several views of the drawings, the electrode positioning suit is shown and is generally indicated as 10. The suit 10 is preferably made of a flexible, elastomeric material such as neoprene, spandex, lycra, etc. In a preferred embodiment, the suit 10 is structured to resemble a wet suit of the type commonly used in scuba diving and surfing. Alternatively, the suit 10 may be fabricated of a thinner elastomeric material such as spandex or lycra, similar to an aerodynamic ski suit 10 be structured to fit somewhat snug to the body, particularly at the areas of the muscle groups and/or nerves being treated.

The suit 10 may be manufactured as a one-piece bodysuit, including an upper body portion 12 and a lower body portion 14. Alternatively, the suit 10 may be manufactured as a two-piece garment, with the upper body portion 12 being separate from the lower body portion 14. Further, it is contemplated that the suit 10 of the present invention may be structured as several pieces to cover a particular area of the body, such as one or both legs, the abdomen, the chest and/or one or both arms. In this particular embodiment, the suit 10 is manufactured as a wrap or sleeve, which covers the specific area of the body to be treated.

The suit 10 is specifically structured to be used in conjunction with an electrical muscle and or nerve stimulation device. More particularly, the suit 10 is structured to advantageously hold and position electrode pads at predetermined positions on the body corresponding with specific muscle groups to be treated by electrical stimulation impulses. To accomplish this, the electrode pads 20 are placed at positions 30 strategically located on the suit at locations which correspond to the particular muscle groups and/or nerve areas which can be selectively treated. Wiring 32 connecting to the electrode pads 20 are routed through seams 38 on the arms, legs, and torso of the suit 10. In a preferred embodiment, the wiring exits at the waist of the suit in a bundle and terminates at an interface 40. The interface 40 is specifically adapted for connection with the output connectors on a TENS unit or EMS unit.

To facilitate ease of placement and removal of the suit 10 on the body, zippers 50 are provided on the arms and legs. The waist of the suit may further be provided with a zipper to permit separation of the upper body portion 12 from the lower body portion 14. Separation of the upper and lower body portions enables the user to wear either just the upper body portion or the lower body portion when targeting specific muscle groups. More particularly, the user may treat the muscle groups of the upper body during one session, while wearing just the upper body portion of the suit, and then during a different session, treat the lower muscles in the legs wearing just the lower portion 14.

The interface is specifically structured to permit selective directing of electrical signals to specific electrode pads on the body suit, thereby targeting specific muscle groups. To simplify identification of the electrode pads associated with each muscle group, the wires connecting between the interface and the electrodes may be color-coded. Thus, for example, the wires leading to the electrodes on the legs may be color-coded blue. Further, electrodes positioned on the abdomen muscles may be green, while the electrodes connecting to the chest may be colored black. Color coding the wires in this manner enables the user to identify the particular connection of the interface and electrode pads with the output connectors of the TENS or EMS unit. This permits operation of the TENS or EMS unit to deliver electrical signals to one or more selected color coded wiring groups, thereby targeting specific muscle groups for stimulation. It should be noted that the EMS unit used in conjunction with the suit of the present invention may be adapted to allow selective control of electrical signal pulse intensity for each muscle group. By varying the duration and intensity of the electrical pulse, each muscle group can be specifically treated in accordance with the needs of the user.

While the invention has been shown and described in connection with preferred embodiment thereof, it is recognized that departures from the disclosure are contemplated within the spirit and scope of the invention.

What I claim as my invention. A body suit or individual pieces comprising:

1. An elastomeric garment holding pre-positioned electrodes for EMS/TENS users.

2. A elastomeric garment containing the wiring within to power the EMS/TENS electrodes.

3. A single interface for said electrodes and EMS/TENS control signal devices.
